(19) United States
(12) Patent Application Publication
(10) Pub. No.: US 2008/0281207 A1
(43) Pub. Date: Nov. 13, 2008

(54) Image acquisition through filtering in multiple endoscope systems

(75) Inventor: Richard S. Johnston, Sammamush, WA (US)

Correspondence Address: BLAKELY SOKOLOFF TAYLOR & ZAFMAN LLP, 1279 OAKMEAD PARKWAY, SUNNYVALE, CA, 94085-4040, US

(73) Assignee: University of Washington

(21) Appl. No.: 11/801,222

(22) Filed: May 8, 2007

(51) Int. Cl. A61B 1/06; A61B 6/00

(52) U.S. Cl. 600/476; 600/101

(57) ABSTRACT

A method of one aspect may include illuminating a surface with a broad bandwidth light from a first endoscope. The broad bandwidth light typically has a bandwidth of at least 200 nanometers (nm). A beam that includes at least one narrow bandwidth light may be scanned over the surface with a second, scanning beam endoscope. The narrow bandwidth light typically has a bandwidth of less than 3 nm. During the scanning, light that has been backscattered from the surface may be collected with the scanning beam endoscope. The collected backscattered light may be filtered with at least one narrow bandwidth band-pass optical filter. A band-pass bandwidth of the filter may be no more than 15 nm and may at least partially overlap the bandwidth of the narrow bandwidth light. The filtered backscattered light may be detected with a photodetector.

### BACKGROUND

1. Field

Embodiments of the invention relate to endoscopes. In particular, embodiments of the invention relate to filtering during image acquisition in multiple endoscope systems.

2. Background Information

Endoscopes are instruments or devices that may be inserted into a patient and used to look inside a body cavity, lumen, or otherwise look inside the patient.

One type of endoscope is a scanning beam endoscope. The scanning beam endoscope may scan a beam or illumination spot over a surface to be viewed. Backscattered light from the illumination spot may be detected by the scanning beam endoscope at different times during the scan in order to construct an image of the surface.

Another type of endoscope is a conventional, non-scanning beam endoscope. Such endoscopes may flood the whole surface to be viewed with a bright white or near white light, for example, provided through one or more generally large multimode optical fibers. Backscattered light may be collected from the whole surface in parallel, and an image may be constructed. In some such endoscopes, a light detector array, for example a charge-coupled device, may be included at a distal tip of the endoscope to detect the backscattered light. In other endoscopes, numerous optical fibers, each corresponding to a pixel in the image, may be used to collect and return the backscattered light to a base station. In the base station, the light may be detected with a light detector array, or otherwise used to construct the image.

Multiple endoscopes are occasionally used in combination. By way of example, a so-called mother endoscope may be used with a so-called daughter or baby endoscope. By way of example, the daughter or baby scope may be used to view areas beyond the reach of the mother endoscope.

### BRIEF DESCRIPTION OF THE SEVERAL VIEWS OF THE DRAWINGS

The invention may best be understood by referring to the following description and accompanying drawings that are used to illustrate embodiments of the invention. In the drawings:

FIG. 1 is a block flow diagram of a method performed in a dual endoscope system that includes filtering backscattered light collected by a scanning beam endoscope, according to embodiments of the invention.

FIG. 2 is a block diagram of a dual endoscope system including at least one narrow bandwidth band-pass optical filter, according to embodiments of the invention.

FIG. 3 is a block diagram of a first base station that includes at least one narrow bandwidth band-reject optical filter, according to one or more embodiments of the invention.

FIG. 4 is a block diagram of an example of a second base station for a full-color scanning beam endoscope, where the base station includes a plurality of narrow bandwidth light sources and a plurality of corresponding narrow bandwidth band-pass optical filters, according to embodiments of the invention.

FIG. 5 is a block diagram of an example of a first base station that includes a plurality of narrow bandwidth band-reject optical filters, according to embodiments of the invention.

### DETAILED DESCRIPTION

In the following description, numerous specific details are set forth. However, it is understood that embodiments of the invention may be practiced without these specific details. In other instances, well-known circuits, structures and techniques have not been shown in detail in order not to obscure the understanding of this description.

Challenges may present themselves when certain types of endoscopes, such as, for example scanning beam endoscopes, are used in combination with other endoscopes, such as, for example, the aforementioned conventional endoscopes. For example, if the surface over which the beam or illumination spot from the scanning beam endoscope is scanned is concurrently illuminated with light from another endoscope, then some of the light from the other endoscope may be reflected or otherwise backscattered and collected by the scanning beam endoscope. This light from the other endoscope is generally unwanted and may represent noise, which may tend to reduce the contrast or otherwise adversely affect the quality of images acquired using the scanning beam endoscope.

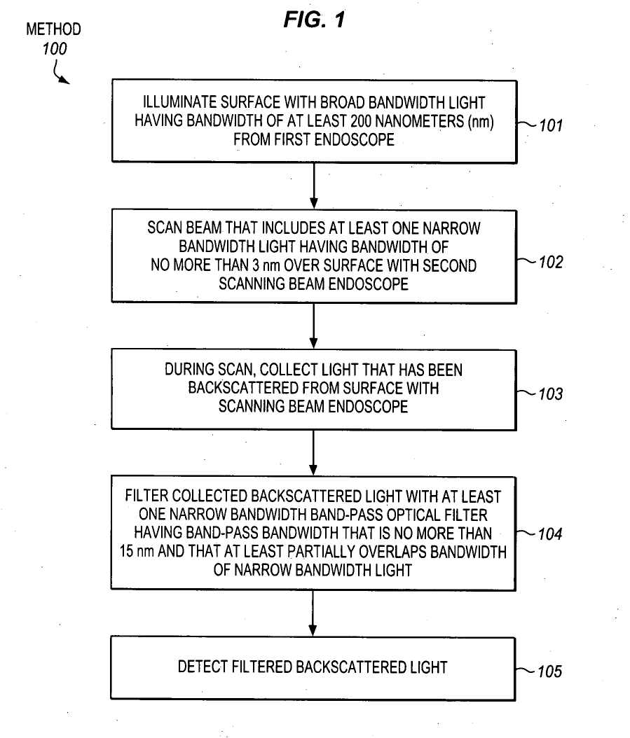

The inventors have conceived of improved systems and methods of imaging in multiple endoscope systems. FIG. 1 is a block flow diagram of a method 100 of imaging in a dual endoscope system that includes filtering backscattered light collected by a scanning beam endoscope in order to remove at least some light originating from another endoscope, according to embodiments of the invention.

Initially, a surface to be imaged may be illuminated with a broad bandwidth light from a first endoscope, at block 101. At least partially concurrently, a beam that includes at least one narrow bandwidth light may be scanned over the surface with a second, scanning beam endoscope, at block 102. In embodiments of the invention, the broad bandwidth light may be white light or near white light typically having a bandwidth of at least 200 nm that floods the surface, whereas each of the at least one narrow bandwidth light typically has a bandwidth of less than about 3 nm.

During the scan of the beam over the surface, light reflected or otherwise backscattered from the surface may be collected with the scanning beam endoscope, at block 103. Note that the collected light may include not only backscattered narrow bandwidth light from the beam from the scanning beam endoscope, but also backscattered broad bandwidth light from the first endoscope. As previously mentioned, this light from the first endoscope is generally unwanted and may represent noise, which may tend to reduce the contrast or otherwise adversely affect the quality of images acquired using the scanning beam endoscope.

Significantly, in embodiments of the invention, the collected backscattered light may be filtered with at least one narrow bandwidth band-pass optical filter, at block 104. For brevity, the narrow bandwidth band-pass optical filter may also be referred to herein simply as a band-pass filter. In embodiments of the invention, each of the at least one band-pass filter may have a band-pass bandwidth that is not more than 15 nm, not more than 10 nm, or not more than 5 nm, and at least 0.1 nm. In embodiments of the invention, each of the at least one band-pass filter may have a bandwidth that at least partially overlaps, or in some cases substantially overlaps, or completely encompasses, a bandwidth of a corresponding one of the at least one narrow bandwidth light.

Advantageously, in various embodiments, the filtering with the band-pass filter may reject or reduce at least some, or most, or substantially all, of the backscattered broad bandwidth light from the first endoscope that is collected by the scanning beam endoscope. At the same time, due to the overlap, at least some, or most, or substantially all, of the backscattered narrow bandwidth light from the beam may pass right through the band-pass filter. Furthermore, the filtering with the band-pass filter may help to reject ambient light (i.e., not necessarily from the first endoscope), or other light besides backscattered light from the beam, which becomes backscattered and collected by the scanning beam endoscope.

Then, the filtered backscattered light may be detected, for example with one or more photodetectors, at block 105. This detected light may be used to construct images of the surface over which the beam is scanned. Advantageously, the rejection or reduction of the broad bandwidth and/or ambient lights by the band-pass filter, may help to improve the contrast and quality of images acquired using the scanning beam endoscope.

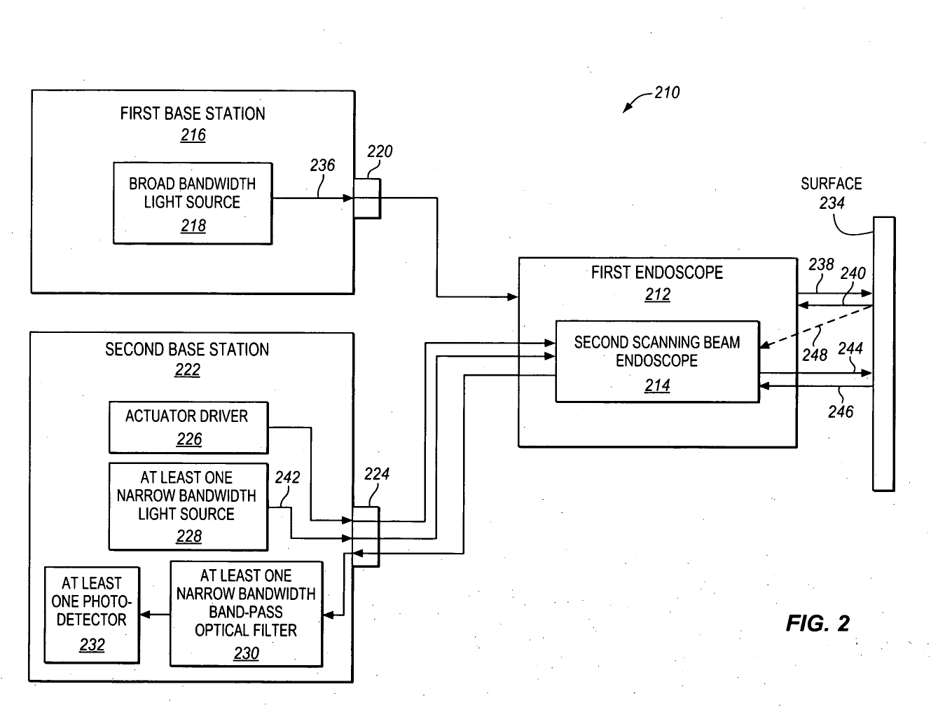

FIG. 2 is a block diagram of a dual endoscope system 210 including at least one narrow bandwidth band-pass optical filter 230, according to embodiments of the invention. The system includes a first endoscope 212, a first base station 216, a second endoscope 214, and a second base station 222.

As is known, endoscopes represent instruments or devices to be inserted into a patient to look inside a body cavity, lumen, or otherwise look inside the patient. Examples of suitable types of endoscopes include, but are not limited to, bronchoscopes, colonoscopes, gastroscopes, duodenoscopes, sigmoidoscopes, thorascopes, ureteroscopes, sinuscopes, boroscopes, and thorascopes, to name just a few examples.

In the illustration, the first and second endoscopes are arranged or configured as mother and daughter endoscopes, respectively, although this is not required. By way of example, the daughter scope may be inserted or otherwise introduced through an internal working channel of the mother scope prior to, or during use. Alternatively, the second scope may be configured as the mother scope, and the first scope may be configured as the daughter scope. As yet another option, the first and second scopes may simply be used in the same area but not configured as mother and daughter.

The first base station has a first connector interface 220 to allow the first endoscope to be connected. The first base station also has a broad bandwidth light source 218. Conventional broad bandwidth light sources used in endoscopes are suitable. The broad bandwidth light source may provide a broad bandwidth light to the first endoscope through the first connector interface. In embodiments of the invention, the broad bandwidth light typically has a bandwidth of at least 200 nanometers (nm).

The second base station similarly has a second connector interface 224 to allow the second endoscope to be connected. The second base station also has at least one narrow bandwidth light source 228. In embodiments of the invention, the second base station may include multiple narrow bandwidth light sources. Examples of suitable narrow bandwidth light sources include, but are not limited to, lasers, laser diodes, vertical cavity surface-emitting lasers (VCSELs), other light emitting devices known in the arts, and combinations thereof. Each narrow bandwidth light source may provide a corresponding narrow bandwidth light to the second endoscope through the second connector interface. In embodiments of the invention, each narrow bandwidth light typically has a bandwidth of less than about 3 nm.

In embodiments of the invention, the second base station may have at least one narrow bandwidth band-pass optical filter 230. In embodiments of the invention, the second base station may have multiple such band-pass filters. As shown, each of the band-pass filters may be optically coupled with the second connector interface, in an optical path of light returned by the scanning beam endoscope through the connector interface. As is known, a band-pass filter may pass wavelengths with a specified, continuous band-pass bandwidth, while rejecting or attenuating wavelengths above and below this band-pass bandwidth.

In various embodiments of the invention, each of the band-pass filters may have a band-pass bandwidth that is not more than 15 nm, not more than 10 nm, or not more than 5 nm, and at least 0.1 nm. In embodiments of the invention, each of the band-pass filters may have a bandwidth that at least partially overlaps, or in some cases substantially overlaps, or completely encompasses, a bandwidth of a corresponding narrow bandwidth light from the at least one narrow bandwidth light source 228. Generally, the greater the amount of overlap the greater the proportion of the collected backscattered narrow bandwidth light that is passed through the filter. A smaller band-pass bandwidth also generally provides a greater reduction of the broad bandwidth light and ambient light.

The second base station also includes at least one photodetector 232 that is optically coupled with an output of the band-pass filter. Examples of suitable types of photodetectors include, but are not limited to, photodiodes, photomultiplier tubes, phototransistors, other photodetectors known in the arts, and combinations thereof. The photodetector may detect filtered light passed through the band-pass filter. Alternatively, rather than including the photodetector and the band-pass filter in the base station, these components may optionally be included in the scanning beam endoscope.

The second base station also includes an actuator driver 226. The actuator driver may provide actuator drive signals to the scanning beam endoscope through the connection interface. The actuator drive signals may actuate a single cantilevered optical fiber, moveable reflector, or other scanning optical element (not shown) of the scanning beam device.

In use, the endoscopes may be positioned near a surface 234. The broad bandwidth light source may provide broad bandwidth light 236 to the first endoscope. The first endoscope may illuminate the surface with broad bandwidth light 238. Backscattered light 240 may be collected by the first endoscope and used to construct an image.

Concurrently with the illumination of the surface, each of the at least one narrow bandwidth light sources may provide narrow bandwidth light 242 to the scanning beam endoscope. The actuator driver may provide actuator drive signals to the scanning beam endoscope. The actuator drive signals may cause the scanning beam endoscope to scan a beam or illumination spot 244, which includes each of the at least one narrow bandwidth light, over the surface in a spiral, propeller, raster, or other scan pattern. In embodiments of the invention, during the scan a single cantilevered optical fiber of the scanning beam endoscope may be vibrated close to or within a Q-factor of its resonance frequency. Further background information on such scanning, if desired, is available in U.S. Patent Application 20060138238, entitled “METHODS OF DRIVING A SCANNING BEAM DEVICE TO ACHIEVE HIGH FRAME RATES”, by Richard S. Johnston et al.

The scanning beam endoscope may collect a backscattered portion 246 of the beam or illumination spot. Typically, the scanning beam endoscope also collects a backscattered portion 248 of the broad bandwidth light. In addition, ambient light may potentially be collected. The collected backscattered light may be returned to the second base station and filtered by the band-pass filter as described elsewhere herein. The filtered light may be provided to the photodetector. An image of the surface may be constructed based on the detected light. Advantageously, since the filtering removes at least some, much, or most of the broad bandwidth light and/or ambient light, image contrast and quality may be improved.

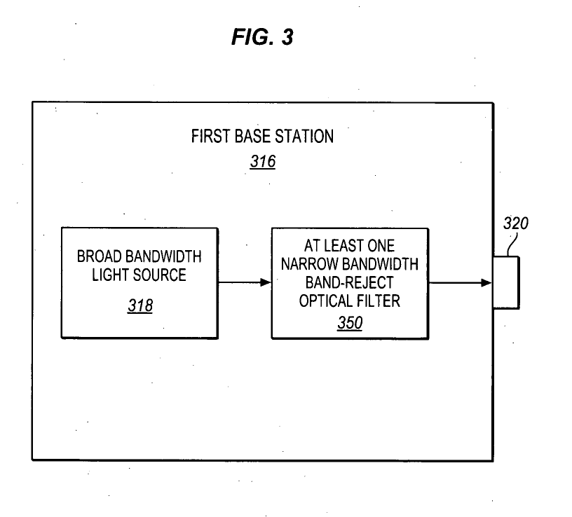

Note however, that a fraction of the broad bandwidth light and/or ambient light may have a bandwidth that overlaps with the band-pass bandwidth and may tend to pass right through the band-pass filter. FIG. 3 is a block diagram of a first base station 316 that includes at least one narrow bandwidth band-reject optical filter 350, according to one or more embodiments of the invention. The first base station may otherwise be similar to, or in-some cases the same as, the first base station 216 shown in FIG. 2.

The base station includes a connector interface 320 to allow an endoscope to be connected. The base station also includes a broad bandwidth light source 318 to provide a broad bandwidth light to the endoscope through the connector interface. As before, in embodiments of the invention, the broad bandwidth light typically has a bandwidth of at least 200 nm.

The base station also includes at least one optional narrow bandwidth band-reject optical filter 350. For brevity, the narrow bandwidth band-reject optical filter may also be referred to herein simply as a band-reject filter. Band-reject filters are also occasionally known in the arts as notch filters. In embodiments of the invention, the base station may include multiple band-reject filters.

The band-reject filter is disposed or positioned in an optical path of the broad bandwidth light. As shown, the band-reject filter may be coupled between the broad bandwidth light source and the connector interface. Alternatively, the band-reject filter may be included in the connector interface or in the first endoscope.

The band-reject filter may receive and filter the broad bandwidth light before it is used to illuminate a surface during acquisition of an image. As is known, a band-reject filter may reject wavelengths within a specified band-reject band, while passing out-of-band wavelengths.

In various embodiments of the invention, the band-reject bandwidth may at least partially overlaps, or in some cases substantially overlaps, or completely encompasses, the bandwidth of the narrow bandwidth band-pass optical filter 230 and/or the bandwidth of the narrow bandwidth light from the narrow bandwidth light source 228 of FIG. 2. Generally, the greater the amount of overlap, the greater the proportion of the broad bandwidth light that is capable of passing through the band-pass filter which will be removed by the band-reject filter.

In embodiments of the invention, the band-reject bandwidth may be sufficiently large to remove a significant portion, most, or substantially all of the broad bandwidth light that would tend to pass through the narrow bandwidth band-pass optical filter, while sufficiently small to avoid significantly altering the whiteness or optical characteristics of the broad bandwidth light. In various embodiments of the invention, the band-reject filter may have a band-reject bandwidth that is not more than 30 nm, not more than 20 nm, not more than 15 nm, not more than 10 nm, not more than 5 nm, or about 1 to 3 nm. The band-reject bandwidth may be at least 0.1 nm.

In this way, the band-reject filter may filter out, reject, or otherwise remove, at least a portion of the broad bandwidth light that would otherwise tend to pass right through the band-pass filter 230 in the base station of the scanning beam endoscope. This may further help to improve the contrast or quality of images constructed using the scanning beam endoscope.

A scanning beam endoscope system with a single narrow bandwidth light source and a single corresponding band-pass filter may be useful for acquiring black-and-white or monochrome images. However, in embodiments it may be desirable to acquire color images.

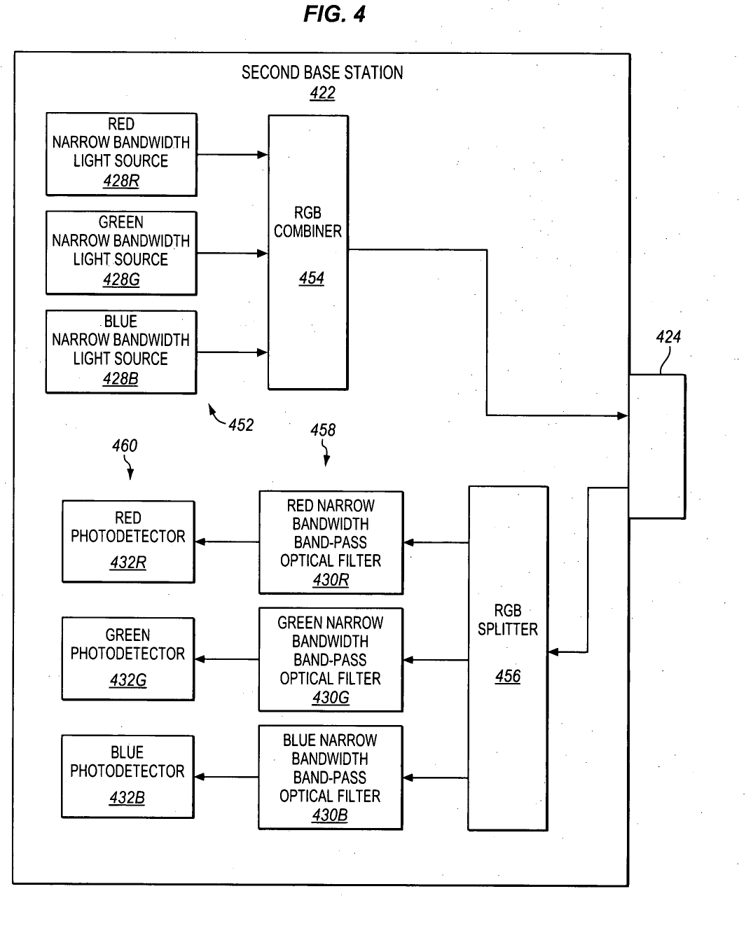

FIG. 4 is a block diagram of an example of a second base station 422 for a full-color scanning beam endoscope, where the base station includes a plurality of narrow bandwidth light sources 428R, 428G, 428B and a plurality of corresponding narrow bandwidth band-pass optical filters 430R, 430G, 430B, according to embodiments of the invention. The second base station may otherwise be similar to, or in some cases the same as, the second base station 222 shown in FIG. 2.

The base station includes a light source 452 that includes a red narrow bandwidth light source 428R, a green narrow bandwidth light source 428G, and a blue narrow bandwidth light source 428B. Strict red, green, and blue colors are not required for the system to construct useful images. As such, as used herein “red”, “green”, and “blue” do not imply any particular average bandwidth, but rather are intended to cover light which is relatively “redish”, “greenish”, or “blueish”. Accordingly, blue may refer to light which is relatively blue-green, for example. Alternatively, the red, green, and blue light sources may optionally be replaced with other suitable light sources, such as, for example, purple, blue-green, magenta, infrared, etc. In various embodiments of the invention, each of the red, green, and blue light sources may have a bandwidth of less than about 3 nm.

A suitable red light source is the 635 nm Model LPS-635 laser diode, which is available from Thorlabs, Inc, of Newton, N.J. A suitable blue light source is the 440 nm Model NDHB510APAEI laser diode, which is available from Nichia Corporation, of Tokyo, Japan. A suitable green light source is a BWN-532-20-SMF diode-pumped solid-state laser at 532 nm, which is available from B&W Tech Inc. However, the scope of the invention certainly is not limited to these particular light sources.

Each of the light sources are coupled with a red-green-blue (RGB) combiner 454, for example through a separate single mode optical fiber. The RGB combiner is coupled between the light source and the connector interface and may combine the narrow bandwidth red, green, and blue lights into an RGB illumination light, which may be provided to a connector interface 424 of the base station. An example of a suitable RGB combiner is the 635/532/440 RGB Combiner, which is available from SIFAM Fibre Optics Ltd., of Devon, United Kingdom.

As shown, the base station may also include an optional RGB splitter 456. The RGB splitter is optically coupled with the connector interface to receive backscattered light collected by an endoscope coupled with the connector interface. By way of example, the endoscope may have one or more optical fibers to return the collected backscattered light to the connector interface. The RGB splitter may split the received light into red, green, and blue portions. By way of example, the RGB splitter may include a conventional assembly of focusing optics and dichroic beam splitters.

The base station also includes a filtering system 458 in an optical path of light returned by the endoscope through the connector interface. The filtering system includes a red narrow bandwidth band-pass optical filter 430R, a green narrow bandwidth band-pass optical filter 430G, and a blue narrow bandwidth band-pass optical filter 430B. Each of these filters may be optically coupled with an output of the RGB splitter to receive the respective red, green, and blue portions of the collected backscattered light. Alternatively, in one or more embodiments, the RGB splitter may optionally be omitted. For example, rather than the RGB splitter splitting the light, a first set of one or more optical fibers of the endoscope may be used to convey collected backscattered light to the red filter, a second set may be used to convey collected backscattered light to the green filter, and a third set may be used to convey collected backscattered light to the blue filter. However, this approach may tend to reduce the amount of light detected.

The band-pass bandwidths of each of the red, green, and blue band-pass filters may at least partially overlaps, or in some cases substantially overlaps, or completely encompasses, a bandwidth of a corresponding red, green, and blue light from the light source. In various embodiments of the invention, each of the red, green, and blue band-pass filters may have a band-pass bandwidth that is not more than 15 nm, not more than 10 nm, or not more than 5 nm, and at least greater than 0.1 nm.

An example of a suitable red band-pass filter is 43-082, which is available from Edmund Optics, of Barrington, N.J. An example of a suitable green band-pass filter is 43-070 also available from Edmund Optics. An example of a suitable blue band-pass filter is 43-058 also available from Edmund Optics.

The base station also includes a plurality of photodetectors 460 that are each optically coupled with an output of a respective one of the red, green, and blue band-pass filters. In particular, the base station includes a red photodetector 432R, a green photodetector 432G, and a blue photodetector 432B. An example of a suitable photodetector is H7826 photomultiplier tube module, which is available from Hamamatsu Photonics K.K., of Japan.

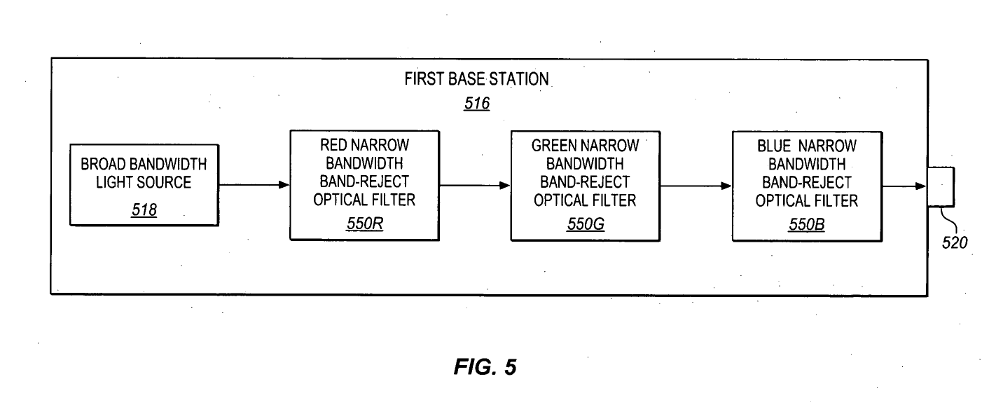

FIG. 5 is a block diagram of an example of a first base station 516 that includes a plurality of narrow bandwidth band-reject optical filters 550R, 550G, 550B, according to embodiments of the invention. The first base station may otherwise be similar to, or in some cases the same as, the first base station 216 shown in FIG. 2.

The base station includes a connector interface 520 and a broad bandwidth light source 518. The base station also includes a red narrow bandwidth band-reject optical filter 550R, a green narrow bandwidth band-reject optical filter 550B, and a blue narrow bandwidth band-reject optical filter 550B. The band-reject filters are optically coupled in series in an optical path of the broad bandwidth light. The illustrated serial order is not required.

The red, green, and blue band-reject filters may respectively remove a narrow bandwidth red, green, and blue portion of the broad bandwidth light. The red, green, and blue portions of the light removed may at least partially or fully overlap with the red, green, and blue lights from the light source 452 of FIG. 4. In one or more embodiments of the invention, the band-reject bandwidths of each of the red, green, and blue band-reject optical filters may be no more than 30 nm, no more than 20 nm, no more than 15 nm, no more than 10 nm, or no more than 5 nm, and at least 0.1 nm.

It is to be appreciated that the band-pass filters disclosed herein are not limited to multiple endoscope systems. The band-pass filters are also useful for reducing unwanted light when a scanning beam endoscope is to be used in a bright light environment and/or used to acquire an image of a lighted or bright surface.

A related approach, which may optionally be used with the approach described herein, is described in co-pending U.S. patent application Ser. No. ______, filed on ______, by Richard S. Johnston et al.

In the description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other.

In the description above, for the purposes of explanation, numerous specific details have been set forth in order to provide a thorough understanding of the embodiments of the invention. The particular embodiments described are not provided to limit the invention but to illustrate it. Embodiments may be practiced without some of these specific details. Furthermore, modifications may be made to the embodiments disclosed herein, such as, for example, to the configurations, functions, and manner of operation, and use, of the components. All equivalent relationships to those illustrated in the drawings and described in the specification are encompassed within embodiments of the invention. The scope of the invention is not to be determined by the specific examples provided above but by the claims below. Further, where considered appropriate, reference numerals or terminal portions of reference numerals may have been repeated among the figures to indicate corresponding or analogous elements, which may optionally have similar characteristics.

It should also be appreciated that reference throughout this specification to “one embodiment”, “an embodiment”, or “one or more embodiments”, for example, means that a particular feature may be included in the practice of the invention. Similarly, it should be appreciated that in the description various features are sometimes grouped together in a single embodiment, Figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. A method comprising:
illuminating a surface with a broad bandwidth light from a first endoscope, the broad bandwidth light having a bandwidth of at least 200 nanometers (nm);
scanning a beam that includes at least one narrow bandwidth light over the surface with a second, scanning beam endoscope, the narrow bandwidth light having a bandwidth of less than 3 nm;
during the scanning, collecting light that has been backscattered from the surface with the scanning beam endoscope;
filtering the collected backscattered light with at least one narrow bandwidth band-pass optical filter, wherein a band-pass bandwidth of the filter is no more than 15 nm and at least partially overlaps the bandwidth of the narrow bandwidth light; and
detecting the filtered backscattered light.

2. The method of claim 1, further comprising constructing an image based on the detected light.

3. The method of claim 1, wherein filtering comprises rejecting a majority of the broad bandwidth light front the first endoscope that is backscattered and collected, while passing a majority of the narrow bandwidth light from the scanning beam endoscope.

4. The method of claim 1, further comprising filtering the broad bandwidth light that is used to illuminate the surface with at least one narrow bandwidth band-reject optical filter having a band-reject bandwidth that is no more than 30 nm and that at least partially overlaps the band-pass bandwidth.

5. The method of claim 4, wherein the band-reject bandwidth is no more than 10 nm and substantially encompasses the band-pass bandwidth.

6. The method of claim 1, wherein illuminating comprises illuminating the surface with a white light, and wherein scanning comprises scanning a beam that includes a plurality of different narrow bandwidth lights each having a bandwidth of less than 3 nm.

7. The method of claim 1:
wherein scanning comprises scanning a beam including a narrow bandwidth red light, a narrow bandwidth green light, and a narrow bandwidth blue light over the surface, each of the narrow bandwidth red, green, and blue lights having a bandwidth of no more than 3 nm; and
wherein filtering comprises filtering the collected backscattered light with a red narrow bandwidth band-pass optical filter, a green narrow bandwidth band-pass optical filter, and
a blue narrow bandwidth band-pass optical filter, each of the red, green, and blue narrow bandwidth band-pass optical filters having a band-pass bandwidth that is no more than 15 nm.

8. The method of claim 7, further comprising, prior to illuminating the surface with the broad bandwidth light:
filtering the broad bandwidth light with a red narrow bandwidth band-reject optical filter having a band-reject bandwidth that is no more than 30 nm;
filtering the broad bandwidth light with a blue narrow bandwidth band-reject optical filter having a band-reject bandwidth that is no more than 30 nm; and
filtering the broad bandwidth light with a green narrow bandwidth band-reject optical filter having a band-reject bandwidth that is no more than 30 nm.

9. The method of claim 1, further comprising introducing the scanning fiber endoscope through a channel of the first endoscope.

10. The method of claim 1, wherein scanning comprises vibrating a single cantilevered optical fiber within a Q-factor of its resonant frequency.

11. An apparatus comprising:
a connector interface to allow an endoscope to be connected;
at least one narrow bandwidth light source to provide at least one narrow bandwidth light to the endoscope through the connector interface, the at least one narrow bandwidth light having a bandwidth of less than 3 nanometers (nm);
at least one narrow bandwidth band-pass optical filter in an optical path of light returned by the endoscope through the connector interface, wherein a band-pass bandwidth of the filter is no more than 15 nm and at least partially overlaps the bandwidth of the narrow bandwidth light; and
a photodetector optically coupled with an output of the filter to detect the filtered light.

12. The apparatus of claim 11, wherein the band-pass bandwidth substantially encompasses the bandwidth of the narrow bandwidth light.

13. The apparatus of claim 11, wherein the at least one narrow bandwidth light source comprises a plurality of narrow bandwidth light sources, and wherein the at least one narrow bandwidth band-pass optical filter comprises a plurality of narrow bandwidth band-pass optical filters, and wherein each of the filters at least partially overlap in bandwidth with light from one of the light sources.

14. The apparatus of claim 11:
wherein the at least one narrow bandwidth light source comprises,
a first narrow bandwidth light source to provide a first narrow bandwidth light having a first bandwidth of no more than 3 nm,
a second narrow bandwidth light source to provide a second narrow bandwidth light having a second bandwidth of no more than 3 nm, and
a third narrow bandwidth light source to provide a third narrow bandwidth light having a third bandwidth of no more than 3 nm; and
wherein the at least one narrow bandwidth band-pass optical filter comprises,
a first narrow bandwidth band-pass optical filter having a band-pass bandwidth of no more than 15 nm and that partially overlaps the first bandwidth,
a second narrow bandwidth band-pass optical filter having a band-pass bandwidth of no more than 15 nm and that partially overlaps the second bandwidth, and
a third narrow bandwidth band-pass optical filter having a band-pass bandwidth of no more than 15 nm and that partially overlaps the third bandwidth.

15. The apparatus of claim 11, further comprising the endoscope connected to the connector interface, wherein the endoscope comprises a single cantilevered optical fiber and an actuator to vibrate the single cantilevered optical fiber within a Q-factor of its resonant frequency.

16. An apparatus comprising:
a connector interface to allow an endoscope to be connected;
a light source to provide light to the endoscope through the connector interface, the light source including,
a red narrow bandwidth light source to provide a narrow bandwidth red light having a bandwidth of no more than 3 nanometers (nm),
a green narrow bandwidth light source to provide a narrow bandwidth green light having a bandwidth of no more than 3 nm, and
a blue narrow bandwidth light source to provide a narrow bandwidth blue light having a bandwidth of no more than 3 nm;
a filtering system in an optical path of light returned by the endoscope through the connector interface to optically filter the returned light, the filtering system including,
a red narrow bandwidth band-pass optical filter having a band-pass bandwidth that is no more than 15 nm and that at least partially overlaps the bandwidth of the narrow bandwidth red light,
a green narrow bandwidth band-pass optical filter having a band-pass bandwidth that is no more than 15 nm and that at least partially overlaps the bandwidth of the narrow bandwidth green light, and
a blue narrow bandwidth band-pass optical filter having a band-pass bandwidth that is no more than 15 nm and that at least partially overlaps the bandwidth of the narrow bandwidth blue light; and
a plurality of photodetectors each optically coupled with an output of one of the red, green, and blue narrow bandwidth band-pass optical filters.

17. The apparatus of claim 16, wherein the band-pass bandwidths of the red, green, and blue filters substantially encompass the respective bandwidths of the red, green, and blue lights.

18. The apparatus of claim 16, further comprising:
a combiner coupled between the light source and the connector interface to combine the red, green, and blue lights; and
a splitter coupled between the connector interface and the red, green, and blue filters to split the returned light into red, green, and blue portions that are provided to the respective red, green, and blue filters.

19. The apparatus of claim 16, further comprising the endoscope connected to the connector interface, wherein the endoscope comprises a single cantilevered optical fiber and an actuator to vibrate the single cantilevered optical fiber within a Q-factor of its resonant frequency.

20. An apparatus comprising:
a connector interface to allow an endoscope to be connected;
a broad bandwidth light source to provide a broad bandwidth light to the endoscope through the connector interface, the broad bandwidth light having a bandwidth of at least 200 nanometers (nm);
at least one narrow bandwidth band-reject optical filter in an optical path of the broad bandwidth light, the filter having a band-reject bandwidth of no more than 30 nm.

21. The apparatus of claim 20, wherein the filter is coupled between the connector interface and the light source.

22. The apparatus of claim 20, wherein the filter has a band-reject bandwidth of no more than 10 nm.

23. The apparatus of claim 20, wherein the light source comprises a white light source to provide white light, and wherein the band-reject bandwidth is to reject a colored light selected from red light, green light, and blue light.

24. The apparatus of claim 20, wherein the at least one narrow bandwidth band-reject optical filter comprises:
a first narrow bandwidth band-reject optical filter having a band-reject bandwidth that is no more than 20 nm;
a second narrow bandwidth band-reject optical filter having a band-reject bandwidth that is no more than 20 nm; and
a third narrow bandwidth band-reject optical filter having a band-reject bandwidth that is no more than 20 nm.

25. The apparatus of claim 24, wherein the first filter comprises a red narrow bandwidth band-reject optical filter, wherein the second filter comprises a green narrow bandwidth band-reject optical filter, wherein the third filter comprises a blue narrow bandwidth band-reject optical filter.
